(19) United States
(12) Patent Application Publication
(10) Pub. No.: US 2024/0198003 A1
(43) Pub. Date: Jun. 20, 2024

(54) HOUSING COMPONENTS AND MEDICAMENT CONTAINER FOR AN INJECTION DEVICE

(71) Applicant: Sanofi, PARIS (FR)

(72) Inventors: Beate Franke, Frankfurt am Main (DE); Michael Helmer, Frankfurt am Main (DE)

(21) Appl. No.: 18/287,256

(22) PCT Filed: May 2, 2022
(86) PCT No.: PCT/EP2022/061648

§ 371 Date: Oct. 17, 2023

(30) Foreign Application Priority Data
May 3, 2021 (EP) 21315078.2

(51) Int. Cl. A61M 5/24; A61M 5/31

(52) U.S. Cl. CPC A61M 5/24; A61M 5/3129; A61M 2005/2403; A61M 2005/3142; A61M 2205/19

(57) ABSTRACT

The present disclosure relates to a housing of a drug delivery device, the housing comprising: a first housing component comprising a first connecting end provided with a fastening element, the first housing component configured to accommodate a medicament container, the medicament container comprising a mechanical coding and an alignment element, a second housing component comprising a second connecting end provided with a counter fastening element complementary shaped to the fastening element, the second connecting end being connectable to the first connecting end, a counter alignment element complementary shaped to the alignment element and provided in or on the first housing component, a mechanical counter coding provided in or on the second housing component, wherein when the mechanical coding does not match the mechanical counter coding, the mechanical coding and the mechanical counter coding being operable to prevent an engagement of the fastening element with the counter fastening element.

### CROSS REFERENCE TO RELATED APPLICATIONS

The present application is the national stage entry of International Patent Application No. PCT/EP2022/061648, filed on May 2, 2022, and claims priority to Application No. EP21315078.2, filed on May 3, 2021, the entire contents of which are incorporated herein by reference.

### TECHNICAL FIELD

The present disclosure relates to the field of drug delivery devices and systems, particularly to injection devices for injecting a liquid medicament. More particularly, the present disclosure is generally directed to drug delivery devices and systems comprising a multi-component housing, wherein one housing component is configured to accommodate a medicament container, such as a cartridge and wherein another housing component is configured to accommodate a drive mechanism to operably engage with the medicament container for expelling or withdrawing a dose of the medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable sizes. Moreover, a dose setting as well as a dose dispensing procedure must be easy to operate and has to be unambiguous.

### SUMMARY

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g., 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

There is, therefore, a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. Similarly, there is also a general need for a dedicated cartridge that allows the medical delivery device to be used with only an authorized cartridge containing a specific medicament while also preventing undesired cartridge cross use.

With drug delivery devices comprising a multi-component housing, e.g., having a first and a second housing component, it is also desirable to provide a failure safe and well-defined mechanical connection between the housing components, which may be detachably or non-detachably connectable. Here, the present disclosure aims to provide an improvement for connecting and disconnecting first and second housing components of a drug delivery device.

In one aspect the disclosure relates to a housing of the device, in particular to a housing of an injection device. The housing may be implemented as a housing of a pen-type injector. The housing comprises a first housing component. The first housing component comprises a first connecting end. The first connecting end is provided with a fastening element. The first housing component is shaped and configured to accommodate a medicament container or medicament reservoir, e.g., a cartridge. The medicament container is typically filled with a liquid medicament.

The medicament container comprises a mechanical coding. The medicament container further comprises an alignment element. The alignment element is provided in addition to the mechanical coding. In the present context, the terms medicament container and medicament reservoir are used synonymously.

The mechanical coding and the alignment element may be provided on an outside surface of the medicament container. Typically, the mechanical coding and the alignment element are provided on a common sidewall of the medicament container. They may be both provided on an outside surface of a sidewall of the medicament container, e.g., on a barrel of the medicament container.

The housing further comprises a second housing component. The second housing component is sized and configured to accommodate a drive mechanism. Typically, the drive mechanism is implemented to operably engage the medicament reservoir in order to expel or to withdraw one or multiple doses of the medicament from the medicament reservoir. The second housing component comprises a second connecting end.

The second connecting end is provided with a counter fastening element. The counter fastening element is complementary shaped to the fastening element of the first housing component. The second connecting end is connectable to the first connecting end. Typically, the first and the second housing components are mutually connectable by their respective first and second connecting ends. When connecting the first connecting end with the second connecting end the fastening element engages the counter fastening element; and vice versa.

Typically, the first housing component and the second housing component are mutually connectable and fixable by way of the mutual engagement of the fastening element and the counter fastening element.

The housing further comprises a counter alignment element complementary shaped to the alignment element and provided in or on the first housing component. By way of the counter alignment element the medicament container can be aligned with regard to the first housing component, typically, upon insertion of the medicament container into the first housing component. The mutual engagement of the alignment element and the counter alignment element may provide a rotational interlock for the medicament reservoir inside the first housing component. Typically, the first housing component is of tubular shape. It may comprise a tubular-shaped barrel. By way of the counter alignment element engaging the alignment element the medicament reservoir is hindered to rotate relative to the first housing component with regard to the longitudinal axis as an axis of rotation. Upon insertion of the medicament reservoir into the first housing component and by aligning and engaging the alignment element of the medicament reservoir with the counter alignment of the first housing component the medicament reservoir can be rotationally locked to the first housing component.

Furthermore, the housing comprises a mechanical counter coding provided in or on the second housing component. The mechanical counter coding is complementary shaped to the mechanical coding of the medicament reservoir. When the medicament reservoir is arranged in the first housing component and when the mechanical coding does not match the mechanical counter coding, the mechanical coding and the mechanical counter coding are operable to prevent an engagement of the fastening element with the counter fastening element. In other words and with a non-matching pair of a mechanical coding and a mechanical counter coding fastening of the first housing component to the second housing component is effectively blocked and prevented. In this way, an unintended cross use of a wrong medicament reservoir not being intended for coupling with a particular drive mechanism can be effectively prevented.

Due to the mutual engagement of the alignment element of the medicament container with the counter alignment element of the first housing component a rotational interlock between the first housing component and the medicament reservoir can be provided. Furthermore, by way of the mutually engaging alignment element and counter alignment element the medicament container may be only insertable into the first housing component in one or a few predefined or dedicated orientations. Accordingly, the alignment element and the counter alignment element only allow and support one or a few selected or dedicated orientations or position of the medicament container relative to the first housing component in which an insertion of the medicament container into the first housing component is possible and supported.

Hence, the alignment element and the counter alignment element define one or several allowable orientations or position of the mechanical coding relative to the fastening element of the first housing component. In this way and when the fastening element engages the counter fastening element a relative position of the mechanical coding of the medicament container relative to the mechanical counter coding of the second housing component can be defined.

The housing provides a mutual coding and counter coding of a medicament container with a second housing component without providing a direct connection between the medicament container and the second housing component. Typically, the housing is void of a direct interconnection between the medicament container and the second housing component. With some examples the second housing component is exclusively connectable to the first housing component. The medicament container is exclusively insertable and engageable with the first housing component. The first housing component is then connectable and engageable with the second housing component.

Typically, the medicament container and the first housing component are longitudinally engageable, e.g., by mutually corresponding abutment surfaces, that may be e.g., provided at a distal longitudinal end of the first housing component and of the medicament container, respectively. The first housing component with the medicament container preassembled therein may be connectable to the second housing component by an insert motion along the longitudinal direction. With a coding of the medicament container non-matching with the counter coding of the second housing component such a longitudinal assembly motion of the first housing component relative to the second housing component may be effectively blocked or impeded.

With some examples the alignment element of the medicament container may be regarded as or may provide a secondary mechanical coding and the counter alignment element may provide a secondary mechanical counter coding. With some examples the medicament container may be encoded by the alignment element and hence by its secondary mechanical coding. This way, insertion of the medicament container into the first housing component is only allowed and supported when the alignment element matches in size, shape or contour with the counter alignment. With other combinations, wherein when the alignment element does not match the counter alignment element or wherein a number of alignment elements does not match a number of counter alignment elements insertion of the medicament reservoir into the first housing component may be effectively prevented and blocked. In this way, the mutual engagement of the alignment element with the counter alignment element may provide a secondary mechanical coding by way of which insertion of a wrong medicament container into the first housing component can be impeded or blocked.

With some examples, the medicament container may be mechanically encoded in a twofold manner. By way of the alignment element it may be mechanically encoded with the first housing component. By way of the mechanical coding it may be encoded with regard to the second housing component.

The intended and twofold mechanical encoding of the medicament container with two different housing components, namely with the first housing component on the one hand side and with the second housing component on the other hand side, further provides the benefit that the first housing component and the second housing component do not have to be modified with regards to the fastening element and counter fastening element in order to provide a kit of differently encoded housing components. Accordingly, the fastening mechanism by way of which the first housing component is connectable and/or fixable to the second housing component may be implemented identically for a series of differently encoded pairs of medicament containers and second housing components, and only optionally for differently encoded first housing components. This enables and simplifies a mass manufacturing of first and second housing components. Production costs for the first and second housing components can be reduced accordingly.

With some examples and with a kit or set of numerous housing components differently encoded, the first housing component may be always implemented in the same manner. Here, the encoding may be exclusively provided by having a kit of medicament reservoirs that distinguish by way of their mechanical coding. Only the second housing component of the kit or set of differently encoded housings may be then provided with a complementary shaped mechanical counter coding. In this way and for a number of differently encoded housings it may be only the second housing component that varies with regard to the counter coding feature whereas the first housing component may remain substantially unchanged. The first housing component may be void of a mechanical coding or mechanical counter coding. Hence, it may be void of respective counter coding features or coding features.

According to a further example the first connecting end comprises a first sidewall. The second connecting end comprises a second sidewall. One of the first connecting end and the second connecting end comprises an insert. The other one of the first connecting end and the second connecting end comprises a receptacle. The receptacle is complementary shaped to the insert. The insert is insertable into the receptacle along a longitudinal direction for mutually fastening the first housing component and the second housing component. In a final assembly configuration the insert is enclosed by the receptacle. With the medicament container preassembled inside the first housing component the insert is insertable into the receptacle only or exclusively when the mechanical coding of the medicament container matches the mechanical counter coding of the second housing component.

Typically, the first connecting end is a longitudinal connecting end. It may be provided at a proximal longitudinal end of the first housing component. Complementary, the second connecting end may be also implemented as a longitudinal connecting end of the second housing component. It may be provided at a distal longitudinal end of the second housing component. The insert and the receptacle provide a nested arrangement or telescopic arrangement of the first and second housing components at least with regards to their first and second connecting ends. The nested or mutually overlapping insertion of the insert and the receptacle provides a durable, mechanically stable and well-defined connecting interface between the first and second housing components.

Typically, the fastening element is provided on one of the outside surface of the insert and the inside surface of the receptacle. The counter fastening element is provided on the other one of the outside surface of the insert at the inside surface of the receptacle. In this way and when the insert is inserted into the receptacle the fastening element engages the counter fastening element and provides a mechanical fixing of the insert to the receptacle, thereby mechanically locking the first housing component to the second housing component.

The mutual engagement of the fastening element and the counter fastening element may be of non-detachable or of a releasable type. With a non-detachable implementation of the fastening element and the counter fastening element the first housing component is permanently connectable and fixable to the second housing component. Without destroying one of the first housing or the second housing component the mutual fixing cannot be abrogated or released. With a detachable or releasable implementation of the fastening element and the counter fastening element the connection between the first and second housing components can be released. This provides the possibility to replace the medicament container, e.g., when the medicament provided therein has been used up.

According to a further example one of the fastening element and the counter fastening element comprises a fastening projection. The other one of the fastening element and the counter fastening element comprises a complementary-shaped fastening recess. Typically, the fastening projection is a radial fastening projection and the fastening recess is a radial fastening recess e.g., with regard to the tubular shape of the first or second housing components.

The fastening projection may engage the fastening recess upon insertion of the insert into the receptacle. By way of the insert motion of the insert into the receptacle the radial fastening projection engages the complementary shaped radial fastening recess.

With some examples there may be provided a pair of fastening elements and a complementary shaped pair of counter fastening elements. With two pairs of fastening and counter fastening elements the respective fastening elements may be provided on opposite side of the sidewall of the insert or of the receptacle. In this way a twofold mechanical engagement between the insert and the receptacle can be provided. Stability and rigidity of the mutual interconnection between the first and second housing components can be improved.

With further examples there may be even three or more fastening elements on the first housing component complementary shaped with a respective number of three or more counter fastening element provided on the second housing component.

According to another example the fastening projection comprises or constitutes a snap element. The fastening recess comprises or constitutes a respective counter snap element complementary shaped to the snap element. With a radial fastening projection and a radial fastening recess the respective snap element and counter snap element are configured and shaped to be elastically deformable in radial direction. By way of the snap element engaging the complementary-shaped counter snap element an audible and/or haptic feedback can be provided to the user that the fastening element engages the counter fastening element.

The snap element provided with the fastening element and the counter snap element provided with the counter fastening element also provide a well-defined final assembly configuration, in which the fastening element is securely engaged with the counter fastening element.

According to a further example the fastening recess comprises a fastening groove. The fastening groove comprises a first groove portion and a second groove portion. The first groove portion extends along the longitudinal direction. The second groove portion extends along a circumferential direction and merges into the first groove portion. Here, the longitudinal and circumferential direction are defined by the tubular shape of the respective first or second housing component. With some examples the second groove portion extends substantially perpendicular to the first groove portion. This way, the fastening recess is of an L-shaped contour.

The mutual engagement of the fastening element with the counter fastening element may require a purely longitudinally directed insert motion of the insert into the receptacle followed by a rotation of the first housing component relative to the second housing component with the longitudinal axis as an axis of rotation. In this way, there may be provided a two-stage assembly process. During a first stage of mutual assembly the first housing component is subject to a longitudinal sliding movement relative to the second housing component, e.g., until a final insert position of the insert into the receptacle has been reached.

Thereafter and during a second stage of assembly the first housing component has to be rotated relative to the second housing component in order to establish a mutual fastening of the fastening element and the counter fastening element. Typically, the snap element and the counter snap element mutually engage after or during the rotation of the first housing component relative to the second housing component. Starting from the final insert position, which is an intermediate assembly configuration, the first housing component has to be rotated or twisted relative to the second housing component until reaching the final assembly configuration, which is e.g., defined by the mutual engagement of the snap element with the counter snap element, thereby also providing a rotational stop for the first and second housing components, respectively.

According to a further example the first groove portion of the fastening groove adjoins a longitudinal end face of one of the first connecting end or the second connecting end. The second groove portion merges into the first groove portion at a longitudinal distance from the respective longitudinal end face of one of the first connecting end or the second connecting end. Since the first groove portion adjoins a longitudinal end face the fastening projection is allowed to enter and to slide along the first groove portion upon insertion of the insert into the receptacle. When reaching the final insert position or configuration the fastening projection is aligned with the second groove portion and is then allowed to enter the second groove portion and to slide along the second groove portion in circumferential direction when the first housing component is rotated relative to the second housing component. By way of the fastening projection engaging the fastening groove a kind of a bayonet joint between the first and second housing components can be provided and established.

With some examples, the counter snap element is located in the second groove portion. It may be provided on a bottom or on a sidewall or side edge of the second groove portion. For example, the counter snap element may be implemented as a radial recess or radial protrusion in the bottom of the second groove portion. Alternatively, the counter snap element may be implemented as a protrusion protruding inwardly from the sidewall or side edge of the second groove portion. Implementing of the counter snap element inside the second groove portion provides numerous possibilities and options for a rather compact design of the respective counter snap element.

According to a further example the counter snap element is located at a longitudinal end of the second groove portion, which is located opposite the first groove portion. The longitudinal end of the second groove portion may provide or form a dead end of the second groove portion. It may define a first circumferential end of the second groove portion located opposite to a second circumferential end of the second groove portion. Typically, the second circumferential end of the second groove portion may merge with the first groove portion. Here, and since the second groove portion extends in circumferential direction, the longitudinal end of the second groove portion may be located circumferentially and/or tangentially offset from the first groove portion.

According to another example the counter snap element comprises a radial recess in the second groove portion and the snap element comprises a radial protrusion to engage with the radial recess of the counter snap element. Here, and with some examples the snap element may coincide with a radial protrusion of the fastening element. With an alternative example the counter snap element comprises a protrusion in the second groove portion and the snap element comprises a complementary-shaped recess to engage with the radial protrusion of the counter snap element. Here, the counter snap element may comprise a protrusion protruding inwardly either from the bottom of the second groove portion or from a side edge of the second groove portion to engage with a complementary shaped recessed structure of the snap element shaped and configured to slide along the second groove portion.

According to a further example one of the mechanical coding and the mechanical counter coding comprises a radial coding projection. The other one of the mechanical coding and the mechanical counter coding comprises a coding recess complementary shaped to the coding projection. With some examples the radial coding projection is provided on an outside surface of a sidewall of the medicament container. Then, the coding recess is provided on an inside surface of a sidewall of the second housing component, e.g., on an inside surface of the receptacle of the second housing component.

Typically, the mechanical coding comprises one or several coding features. Correspondingly, the mechanical counter coding comprises at least one complementary-shaped mechanical counter coding feature. The mechanical coding may be defined and may distinguish from other mechanical coding by the number of coding features, by the geometric shape of coding features, by the circumferential position of coding features, the circumferential extent of respective coding features, a radial position of coding features, a radial extent of respective coding features and/or by a cross-sectional geometry or shape of the respective coding features in a plane transverse to the longitudinal direction, hence in a plane defined by the radial direction and the circumferential direction with regards to the tubular shape of the first or second housing components.

The mechanical counter coding may be complementary shaped and may be defined accordingly with regard to respective counter coding parameters, e.g., with regard to a number, circumferential position, circumferential extent, radial position, radial extent and/or with regards to a cross-sectional geometry or shape of respective counter coding features in the transverse plane.

With some examples the mechanical counter coding may comprise a radial recess or radial protrusion extending radially at the inside sidewall at a distal end face of the second housing component. Upon insertion of the insert into the receptacle the mechanical counter coding, hence the mechanical counter coding feature may serve and behave as a keyhole or as a key providing a sliding motion of the complementary shaped coding feature of the mechanical coding of the medicament container when correctly preassembled inside the first housing component.

Once the final insert position or final insert configuration has been reached the mechanical coding may have passed the mechanical counter coding in longitudinal direction. Upon reaching the final insert position or insert configuration the mechanical coding may be out of engagement from the mechanical counter coding; and vice versa.

With other examples the mechanical coding and the mechanical counter coding may remain in mutual engagement when reaching the final insert position. The mechanical coding and the mechanical counter coding may also remain in mutual engagement during the second stage of assembly, e.g., when the first housing component is rotated relative to the second housing component.

According to a further example the coding recess comprises a coding groove. The coding groove comprises a first groove portion and a second groove portion. The first groove portion extends along the longitudinal direction. The second groove portion extends along a circumferential direction and merges into the first groove portion. This way, the coding groove may comprise an L-shaped geometry. The coding groove may be void of a through opening extending through any one of the first sidewall or the second sidewall of the respective first housing component or the respective second housing component.

With some examples the second groove portion extends perpendicularly to the first groove portion. Typically, the overall shape of the coding groove is substantially identical to the overall shape of the fastening groove. In this way and as the fastening projection is guided through the first and second groove portions of the fastening groove, the coding projection slides through respective first and second groove portions of the coding groove.

According to a further example the first groove portion of the coding groove adjoins a longitudinal end face of one of the first connecting end or the second connecting end. The second groove portion merges into the first groove portion at a predefined longitudinal distance from the longitudinal end face of one of the first connecting end or the second connecting end. This way, a kind of a bayonet groove with an L-shaped geometry can be provided. By way of the mutual engagement of the coding projection and the coding groove a well-defined and direct mechanical engagement between the medicament container and the second housing component can be provided. In this way, mechanical tolerances between the drive mechanism and the medicament reservoir can be reduced.

With some examples it is even conceivable that the second groove portion is slanted with regard to the circumferential direction. Hence, the second groove portion may comprise a helical shape and may thus comprise a certain, hence non-zero lead. This way, and during a mutual rotation of the first and second housing components by way of which the medicament container rotates in unison with the first housing component relative to the second housing component the mutual engagement of the coding projection with the coding groove may serve to move the medicament reservoir in proximal direction towards the drive mechanism. With some examples the medicament container comprises a tubular-shaped barrel sealed in proximal direction by a bung or stopper movable along the longitudinal direction relative to the sidewall of the barrel, e.g., in order to expel a dose of the medicament from a distal end of the medicament reservoir.

With a helically shaped coding groove provided by at least one of the mechanical coding or the mechanical counter coding at least a slight axial or longitudinal movement of the medicament reservoir relative to the first and/or second housing component and hence relative to the drive mechanism can be easily implemented. This way, it can be provided that upon reaching a final assembly configuration a distal end of the drive mechanism, e.g., a piston rod, gets in direct longitudinal abutment with a proximal end face of the bung or stopper of the medicament reservoir.

In this way, longitudinal tolerances of the drive mechanism or between the drive mechanism and the medicament reservoir can be effectively reduced. Correspondingly, a priming procedure or conducting of an air shot when using the medicament reservoir for the first time may become superfluous and may be effectively circumvented.

With some examples the mechanical coding comprises at least two coding features, e.g., provided at diametrically oppositely located portions of the first or second sidewall. Complementary, the mechanical counter coding comprises respective first and second counter coding features provided at respective diametrically opposite locations of the other one of the first and second sidewall. In this way, there may be provided an at least twofold mechanical engagement between the mechanical coding and the mechanical counter coding. In the event that a longitudinal force effect should be transferred via the mechanical coding engaging the mechanical counter coding, the multiple and simultaneous engagement of at least two coding features with at least two complementary-shaped counter coding features is beneficial in order to prevent asymmetric force effects between the second housing component and the medicament reservoir.

According to a further example the mechanical counter coding comprises at least one counter coding feature and the mechanical counter coding is defined by at least one of a number of counter coding features on the second sidewall, a circumferential position of the counter coding features relative to the counter fastening element, a circumferential extent of counter coding features, a radial extent of counter coding features on the second sidewall and/or by a cross-sectional geometry or cross-sectional shape of counter coding features in a plane transverse to the longitudinal direction.

For a mechanical counter coding matching with a complementary shaped mechanical coding it is required that each one of the above listed coding parameters matches with the respective coding parameter of the mechanical coding. If any one of the above-mentioned counter coding parameters of the mechanical counter coding does not match a respective coding parameter of the mechanical coding, respective coding features non-matching the counter coding features serve to prevent mutual assembly of the medicament container with regards to the second housing component and further serve to prevent mutual assembly of the first housing component and the second housing component of the drug delivery device.

According to another example the mechanical coding may be defined by a combination of a position of the coding feature with regard to the longitudinal direction and an extent of the coding feature in the longitudinal direction. For a coding feature matching with a complementary-shaped counter coding feature it may be required that the longitudinal extent of the radial coding projection matches the longitudinal extent, hence the longitudinally extending width of the second groove portion of the coding groove. Otherwise and if the longitudinal extent of the coding projection is larger than the width or the longitudinal extent of the second groove portion the coding projection is hindered to enter the second groove portion, thereby blocking a rotational movement of the medicament container relative to the first housing component. Since the medicament container is rotationally locked or lockable to the first housing component by the mutual engagement of the alignment element with the counter alignment element the coding feature non-matching with the counter coding feature also prevents a rotation of the first housing component relative to the second housing component towards the final assembly configuration.

With some examples the longitudinal position of the coding feature, hence the longitudinal position of the coding projection may not match the longitudinal position of the second groove portion. Then, it may be possible to insert the coding projection in longitudinal direction into and through the first groove portion of the coding groove but upon reaching a final insert configuration of first and second housing components the coding projection is not properly aligned with the second groove portion. Then, a rotation of the medicament container and hence a rotation of the first housing component relative to the second housing component is effectively blocked and impeded likewise.

With some examples the coding feature and the counter coding feature of a first housing of a kit of housing distinguishes from coding features and counter coding features of another housing by varying the longitudinal position of the second groove portion and the longitudinal position of the coding protrusion correspondingly. Here, an increase of a longitudinal distance of the second groove portion from a longitudinal end of one of the second housing component and the sidewall of the medicament container is accompanied by an increase of the longitudinal extent of the complementary shaped coding projection and by a corresponding decrease of a distance of the coding projection from the other one of the longitudinal end of the second housing component and the sidewall of the medicament container. In this way it can be provided, that one dedicated mechanical coding matches or mates with only one dedicated mechanical counter coding; and vice versa.

According to a further example one of the alignment element and the counter alignment element comprises an alignment projection. The other one of the alignment element and the counter alignment element comprises an alignment recess. The alignment recess is complementary shaped to the alignment projection. Typically, the alignment element is provided on an outside surface of the medicament reservoir. The counter alignment element is provided on an inside surface of the first housing component. The alignment element and the mechanical coding may be located at a common longitudinal position or portion of the sidewall of the medicament container.

With some examples the alignment element and the mechanical coding may be both provided at a proximal end of the medicament container.

With some examples the alignment element may be longitudinally and/or tangentially offset from the mechanical coding.

With some examples the mechanical counter coding is provided or located longitudinally and/or tangentially offset from the counter fastening element.

Typically, the counter coding is separated from the counter fastening element.

With some examples the counter alignment element is provided or located longitudinal and/or tangentially offset from the fastening element.

Typically, the counter alignment element is separated from the fastening element.

By separating the fastening element from the counter alignment element and/or by separating the counter fastening element from the mechanical counter coding there can be provided a kit of differently encoded housings of drug delivery devices that comprise a common fastening structure, e.g., identically shaped and/or identically configured fastening elements and counter fastening elements. Here, different housings of the kit may distinguish only by at least one of a position, a shape, or a configuration of a counter alignment element or a mechanical counter coding.

Typically, the mechanical coding is provided at a proximal end of the medicament container.

The alignment element may be located at a proximal end of the medicament container, in a longitudinal midsection of the medicament container or at a distal end of the medicament container.

The counter alignment element may be provided at a proximal end of the first housing component. Then, the alignment element is also provided at a proximal end of the medicament container. Such an arrangement may be beneficial to provide visual or haptic guidance for inserting the medicament container into the first housing component in the course of the final assembly of the drug delivery device. The counter alignment element is e.g., visible or palpable at the proximal end of the first housing component and may provide a visible or a palpable indication to the user with regards to a correct orientation of the medicament reservoir, so that the medicament reservoir can be correctly inserted into the first housing component along the longitudinal direction, typically from the proximal end of the first housing component towards the distal end of the first housing component.

Preferably, the alignment projection is provided at a proximal end of the sidewall of the medicament reservoir. The alignment recess may be provided at a proximal end of the first housing component. It may be provided at a proximal end of the insert. The alignment recess may be provided as a radial recess on an inside surface of the sidewall of the first housing component. It may adjoin a proximal end face of the sidewall of the first housing component, e.g., it may adjoin a proximal end face of the insert of the first housing component. Providing of the counter alignment element at or near a proximal end of the first housing component is beneficial in that the medicament container can be inserted into the first housing component in any arbitrary orientation. It is only before or upon reaching a preassembly configuration, in which the medicament reservoir is entirely received in the first housing component, that the medicament reservoir has to be oriented in such a way, that the alignment element matches and engages the counter alignment element. For the rest of the insert motion the alignment element and the counter alignment element may have no effect and may not have an impact on the insertion of the medicament container into the first housing component.

According to a further example the alignment projection is a radial projection on one of the sidewall of the medicament container and the sidewall of the first housing component. The alignment recess is a radial recess on the other one of the sidewall of the medicament container and the sidewall of the first housing component.

With other examples it is even conceivable that the alignment projection is a longitudinal projection provided e.g., on one of the distal end of the inside of the first housing component and an outside of the medicament container. Complementary, the alignment recess is a respective longitudinal recess on the other one of a distal end of the medicament container and the distal end of the first housing component. In either way, i.e. by a radial projection matching and mating a radial recess as well as with a longitudinal projection mating a longitudinal recess, a rotational interlock of the medicament container inside the first housing component can be provided. The medicament container is insertable into a preassembly configuration inside the first housing component in only one or a few a dedicated orientations relative to the first housing component.

According to a further example the alignment recess longitudinally adjoins a longitudinal end face of one of the sidewall of the medicament container or the sidewall of the first housing component. With some examples the alignment recess is provided on an inside surface of the sidewall of the first housing component and adjoins a proximal end face of the sidewall of the first housing component. The alignment recesses may be provided as a through recess. With other examples the alignment recess is a blind recess, i.e. a recess having a radial depth that is smaller than the radial thickness of a respective sidewall portion. In the latter case, the alignment recess has only a minimum impact on the stability or rigidity of the respective sidewall of the first housing component.

According to another example the insert comprises a through recess in one of the first sidewall or the second sidewall. The through recess is sized and shaped to receive at least one of the mechanical coding or the mechanical counter coding radially there through. With some examples, the through recess is provided in the first sidewall and comprises a longitudinal slot adjoining a proximal end face of the sidewall of the first housing component. In this way, the mechanical coding provided on the medicament reservoir and located inside the first housing may reach radially through the through recess in order to engage the complementary shaped mechanical counter coding as provided on the inside surface of the second sidewall.

When the first housing component is provided with the insert at the first connecting end the mechanical coding may extend radially outwardly from the medicament reservoir through the through recess to engage with the mechanical counter coding provided at the inside of the sidewall of the receptacle of the second housing component.

With an alternative example, the mechanical counter coding may comprise a radial coding projection extending radially inwardly through the through recess of the first housing component to engage the complementary shaped radial coding recess as provided on the outside surface of the medicament reservoir.

According to another example it is the second housing component that comprises the through recess in the second sidewall. Also here, the through recess is sized and shaped to receive at least one of the mechanical coding or the mechanical counter coding radially there through. With this implementation it may be the first housing component that comprises the receptacle at the first connecting end and the second housing component may comprise the insert provided at the second connecting end. Here, it is again the insert, hence the sidewall of the insert that comprises the through recess enabling a coding projection of one of the mechanical coding and the mechanical counter coding to engage a complementary shaped coding recess of the other one of the mechanical coding and the mechanical counter coding.

According to another example the through recess provides or form a secondary mechanical counter coding complementary shaped to the mechanical coding of the medicament container. When the secondary mechanical counter coding does not match the mechanical coding of the medicament container the mechanical coding and the secondary mechanical counter coding are operable to prevent a complete insertion of the medicament reservoir into the first housing component or to prevent a mutual assembly of the first housing component and the second housing component

For instance and when the insert is provided on the first connecting end the through recess is provided on a sidewall of the insert and forms a secondary mechanical counter coding. Here, insertion of the medicament container in a preassembly configuration inside the first component is only possible when the alignment element matches the counter alignment element and when the mechanical coding matches and mates the through recess and hence the secondary mechanical counter coding.

In this way, it can be effectively prevented, that a medicament reservoir comprising a mechanical coding incompatible with the secondary mechanical counter coding of the first housing component can be inserted into the first housing component. With some examples the through recess is shaped and sized to match the circumferential size of the mechanical coding of the medicament reservoir. With other examples the through recess is larger or much larger than the size or shape of the mechanical coding as provided on the medicament reservoir. In the latter case, and when the through recess comprises a circumferential size or width exceeding the circumferential size or width of the mechanical coding at least two- or even three time the through recess may be suitable and usable for any type of available mechanical coding. Here, it may be exclusively the mutual engagement or non-engagement of the mechanical coding with the mechanical counter coding of the second housing component that prevents mutual assembly of the first and second housing component when the mechanical coding does not match the mechanical counter coding.

According to a further example the through recess adjoins a longitudinal end face of the first connecting end or of the second connecting end. The through recess is open towards the respective longitudinal end face of the respective first or second connecting ends. In this way, the radial coding protrusion is allowed to enter the respective through recess during the first stage of mutual assembly of the first and second housing components, i.e. when the first and the second housing components are subject to a longitudinal sliding movement during the first stage of final assembly.

According to a further example the secondary mechanical counter coding comprises at least one secondary counter coding feature. The secondary mechanical counter coding is defined by at least one of a number of secondary counter coding features on one of the first sidewall or the second sidewall, a circumferential position of secondary counter coding features relative to the counter alignment element and/or relative to the fastening element when the secondary mechanical counter coding is provided on the first housing component, a circumferential position of secondary counter coding features relative to the counter fastening element when the through recesses is provided on the second sidewall, a circumferential extent of the secondary counter coding features, a radial extent of the secondary counter coding features on the first sidewall or second sidewall, and/or a cross-sectional geometry or shape of the secondary counter coding features in a plane transverse to the longitudinal direction.

When any one of the above mentioned secondary mechanical counter coding features does not match the corresponding coding feature of the medicament reservoir, insertion of the medicament reservoir into the first housing component or mutual assembly of first and second housing components is effectively, blocked, prevented or at least impeded.

With some examples the receptacle is provided as a housing insert fixedly attachable or fixedly attached to an elongated housing component, e.g., the first or second housing component of the housing of the drug delivery device. The housing insert may be rotationally and/or longitudinally fixed to the elongated housing component. Insofar all features and benefits as described above in connection with the receptacle equally apply to a housing insert fixedly connectable or fixedly connected to a respective housing component.

According to another aspect the present disclosure relates to a medicament container. The medicament container comprises a barrel sized and configured for insertion into a first housing component of a housing as described above. The medicament container further comprises the alignment element on the barrel which is sized and shaped to engage with the counter alignment element of the first housing component when the barrel is inserted in longitudinal direction into the first housing component. Typically, the medicament container is insertable into the first housing component along a distal direction, hence from the proximal end of the first housing component towards the distal end of the first housing component.

The medicament container further comprises the above-mentioned mechanical coding on the barrel. The mechanical coding is sized and shaped to engage with the mechanical counter coding of the second housing component when the first housing component with the barrel arranged inside is connected to the second housing component. The first housing component with the barrel arranged inside may be regarded as a preassembly configuration of the first housing component. Here, the barrel has reached a final assembly position inside the first housing component. It may partially protrude from the proximal end of the first housing component or it may be entirely received inside the housing component.

Typically, and with further examples the barrel is made of a vitreous material or of a plastic material. The material of the barrel is chemically or pharmaceutically inert. The plastic material may comprise a material composition. The plastic material and/or the material composition may comprise any of cyclic olefin copolymer (COC) and/or cyclo-olefin polymer (COP). The plastic material and/or the composition materials can be configured to have a high double refraction (e.g., optical refractive index higher than 1.5), to provide a high moisture barrier (e.g., moisture absorption smaller than 0.01) and a good material strength (e.g., a charpy impact strength of about 13 to 15). For example, plastic materials, such as COC materials include high purity, high moisture barrier, excellent double refraction, breakage prevention, and low density. Most COC grades can undergo sterilization by gamma radiation, by high temperature steam, or by ethylene oxide. COC also has a very low energy and a nonreactive surface, which can extend shelf life and purity of medications, such as insulin and other protein drugs, stored in medicament containers.

According to a further example at least one of the alignment element or the mechanical coding is integrally formed with the barrel of the medicament container. With some examples, both, the alignment element and the mechanical coding are integrally formed with the barrel. With other examples at least one of the alignment element or the mechanical coding is assembled and fixed to the outside surface of a sidewall of the barrel.

With some examples the medicament container is implemented as a cartridge. The cartridge comprises a distal end is sealed by a pierceable seal, e.g., implemented as a septum. The cartridge is sealed in proximal direction by a piston or bung movably disposed inside the tubular-shaped barrel. The medicament container and hence the barrel provides an interior space filled by the liquid medicament. The interior space is longitudinally confined by the distal seal and the proximally located bung or stopper. In circumferential direction the interior volume is confined by the sidewall of the barrel.

According to further examples the alignment element and the mechanical coding may be provided on a coding ring. The coding ring may be a separate part configured for attachment to the sidewall of the barrel. Typically, the coding ring is configured and sized to form a friction fit or a form fit with the sidewall of the barrel. With some examples the coding ring may be elastically deformable in radial direction so as to enable and to facilitate mounting of the coding ring onto the outside surface of the barrel. The coding ring may be clamped to the barrel. The coding ring may comprise a plastic material. With other examples the coding ring may be made of metal. The alignment element and the mechanical coding may be integrally formed into the coding ring. The at least one of the alignment element or the mechanical coding may comprise a radially raised ridge extending along the longitudinal direction with regard to the tubular shape of the barrel.

With other examples at least one of the alignment element or the mechanical coding are integrally formed with a label or are provided by a label. The label may be provided with an indicator or with a printed surface. The label may be adhesively attached to an outside surface of the sidewall of the barrel. Insofar and by way of attaching a label to the barrel of the medicament container the medicament container can be mechanically encoded and can be provided with the respective alignment element.

According to a further example the mechanical coding comprises at least one coding feature and the mechanical coding is defined by a number of the at least one coding feature on the barrel, a circumferential position of the at least one coding feature relative to the alignment element on the barrel, a circumferential extent of the at least one coding feature, a radial extent of the at least one coding feature on the barrel, and/or a cross-sectional geometry or shape of the at least one coding feature in a plane transverse to the longitudinal direction.

According to a further aspect the present disclosure also relates to an injection device. The injection device comprises a housing as described above and a medicament container as described above. Here, the medicament container comprises the mechanical coding and the alignment element and the medicament container is arranged or is arrangeable inside the first housing component. The injection device further comprises a drive mechanism arranged inside the second housing component. The drive mechanism is typically fixed inside the second housing component.

With other examples the drive mechanism comprises a pump, e.g., a suction pump by way of which a dose of the medicament can be withdrawn from the medicament container by way of suction.

The drive mechanism may be operable to set and to dispense a dose of the medicament from the medicament container. The drive mechanism may be operable to set doses of different size and to conduct repeated and numerous dose setting and dose injection procedures.

According to a further example the medicament container comprises a cartridge, wherein the barrel of the cartridge is sealed towards the proximal direction by a movable bung or stopper arranged inside the barrel. The drive mechanism comprises a piston rod operable to exert a distally directed dispensing force onto the stopper or bung of the cartridge.

According to another aspect the present disclosure relates to a kit of injection devices of the above-described type. The kit of injection devices comprises at least a first injection device as described above and further comprises at least a second injection device as described above. Here, one of the mechanical coding or the mechanical counter coding of the first injection device distinguishes from a respective mechanical coding or mechanical counter coding of the second injection device. The mechanical coding of the first injection device distinguishes from the mechanical coding of the second injection device with regard to at least one of a number of coding features, a circumferential position of coding features relative to the alignment element on the barrel, a circumferential extent of coding features, a radial extent of coding features, or a cross-sectional geometry or shape of coding features in a plane transverse to the longitudinal direction.

Complementary, the counter coding features of the first injection device distinguish from respective counter coding features of the second injection device with regard to at least one of a number of counter coding features, a circumferential position of counter coding features relative to the counter fastening element, a circumferential extent of counter coding features, a radial extent of counter coding features, or a cross-sectional geometry or shape of counter coding features in a plane transverse to the longitudinal direction.

The first injection device is provided with a pair of a coding and a counter coding of a first type.

The second injection device is provided with a pair of a coding and a counter coding of a second type. The coding of the first type is unable to pair or to engage the counter coding of the second type. Vice versa, the counter coding of the first type is unable to pair or to engage with the coding of the second type. In this way the medicament reservoir of the first injection device is unable to pair or to connect with the second housing component of the second injection device. Unintended cross-use of a medicament reservoir with a second housing component and hence with a drive mechanism provided and equipped with different and hence non-matching coding and counter coding can be effectively prevented and impeded.

It is only the coding of the first type which is able and configured to pair or to engage with the counter coding of the first type. The coding of the second type is only and exclusively engageable or connectable to the counter coding of the second type; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl PEPTIDASE-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### DETAILED DESCRIPTION OF THE FIGURES

In the following, numerous examples of injection devices and medicament containers with dedicated or coded housing components will be described in greater detail by making reference to the drawings, in which:

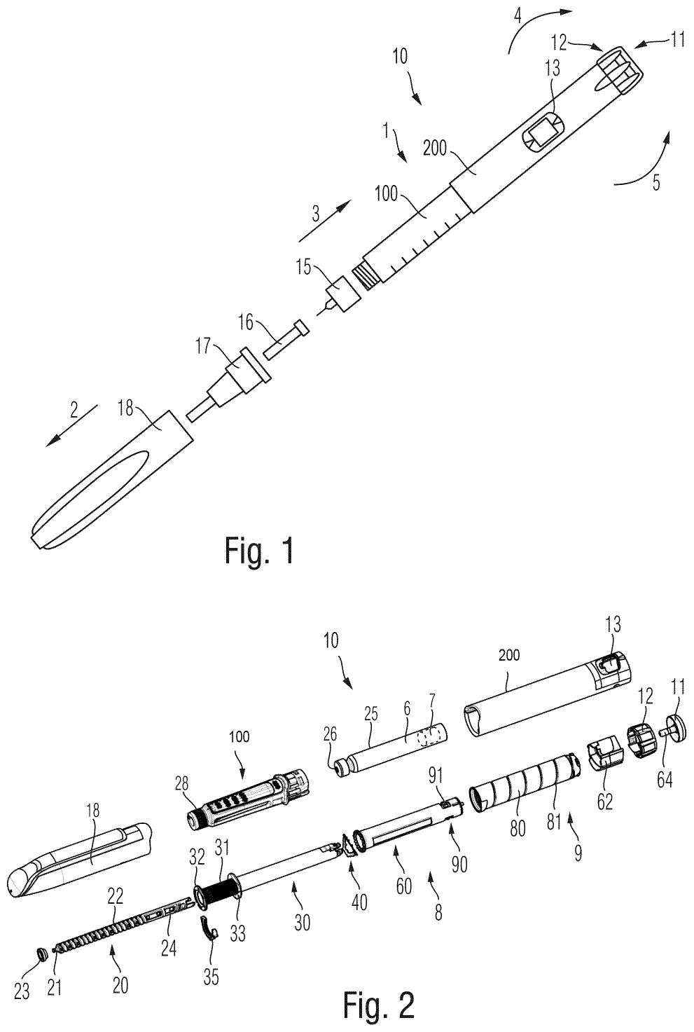

FIG. 1 schematically illustrates an example of a drug delivery device,

FIG. 2 shows an example of an exploded view of the drug delivery device of FIG. 1,

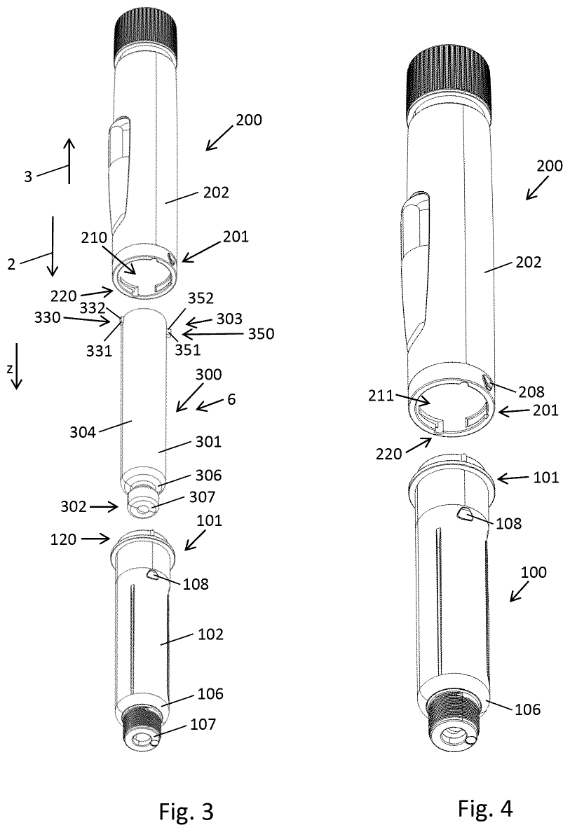

FIG. 3 shows an example of an injection device before insertion of the medicament container into the first housing component,

FIG. 4 shows the injection device of FIG. 3 before connecting first and second housing components,

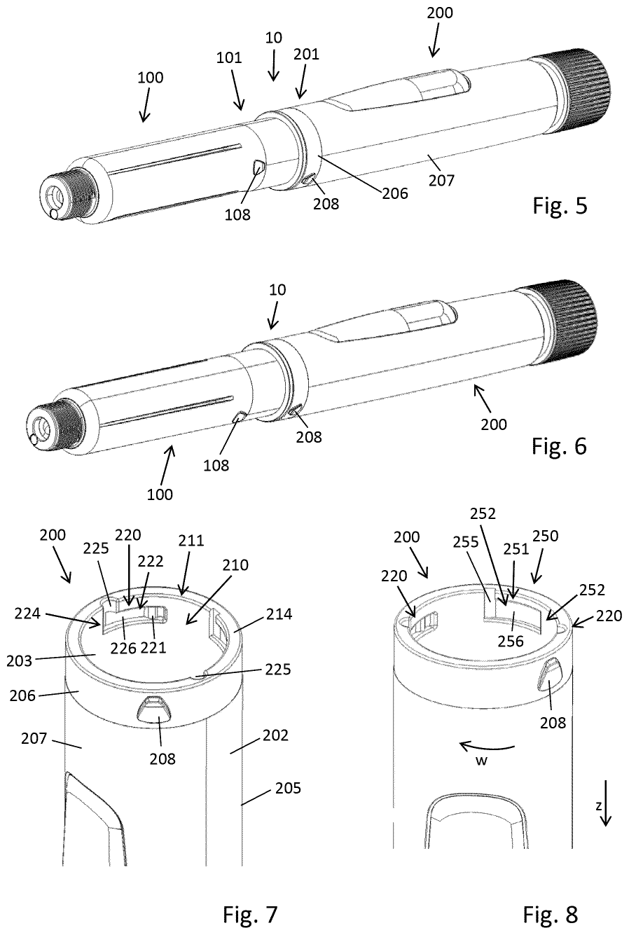

FIG. 5 shows the injection device with the first housing component in the final insert configuration relative to the second housing component,

FIG. 6 shows the injection device in a final assembly configuration,

FIG. 7 is a perspective view of the distal end of the second housing components,

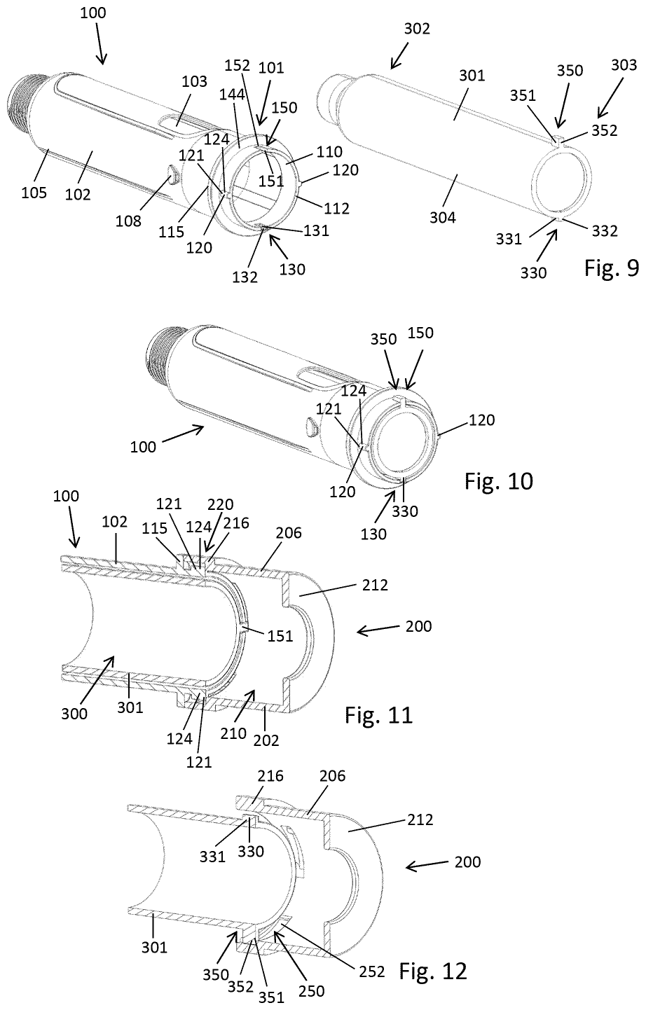

FIG. 8 is a further perspective view of the distal end of the second housing component, FIG. 9 is a perspective view of the first housing component and of the medicament container before insertion into the first housing component,

FIG. 10 shows the housing component and the medicament container in the preassembly configuration,

FIG. 11 schematically illustrates a cross-section of the first housing component connected to the second housing component,

FIG. 12 shows a further cross-section of the medicament reservoir located inside the second housing component,

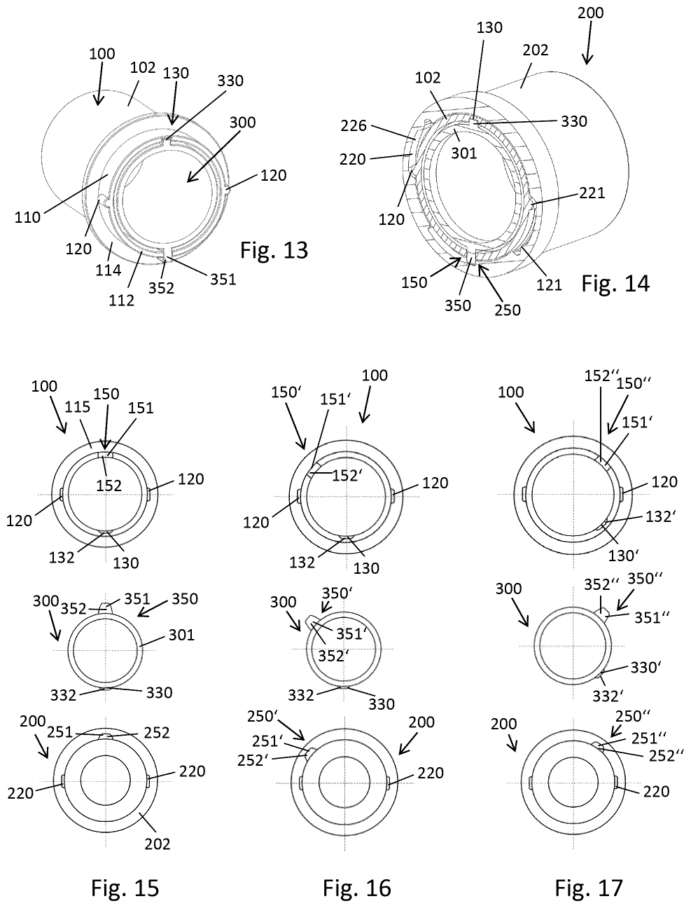

FIG. 13 is an enlarged perspective view of the proximal end of the first housing component with the medicament container assembled therein

FIG. 14 is a cross-section through the first and second housing component when mutually connected,

FIG. 15 shows an example of a first coding and a respective counter coding,

FIG. 16 shows an example of a second coding and a respective counter coding,

FIG. 17 shows another example of a third coding and a respective counter coding,

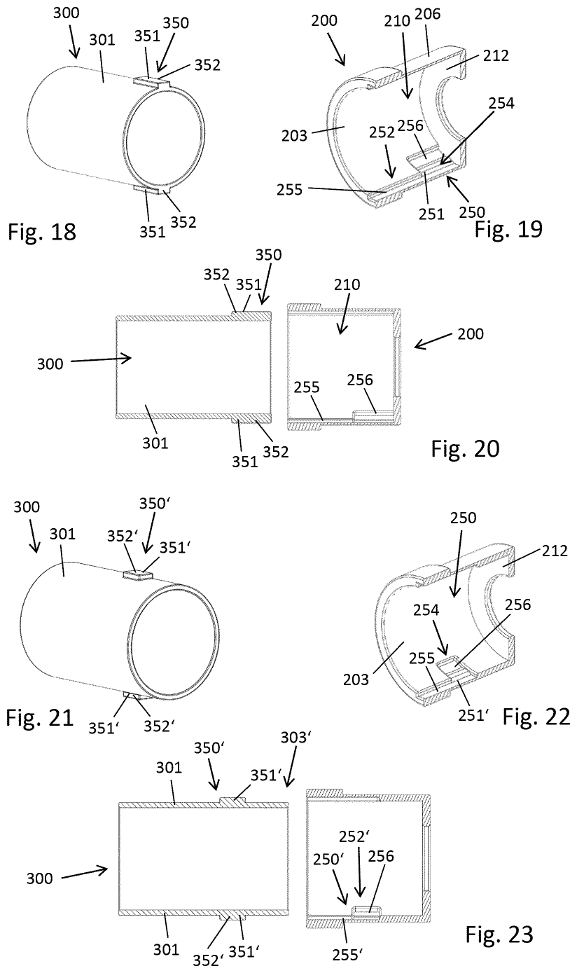

FIG. 18 is a perspective illustration of a mechanical coding of the first type,

FIG. 19 is a perspective and partially cut illustration of a mechanical counter coding of the first type,

FIG. 20 shows the mechanical coding and the mechanical counter coding of FIGS. 18 and 19 in a preassembly configuration,

FIG. 21 shows another example of a mechanical coding,

FIG. 22 shows another example of a mechanical counter coding matching the mechanical coding of FIG. 21,

FIG. 23 shows the mechanical coding and the mechanical counter codings of FIGS. 21 and 22 in a preassembly configuration,

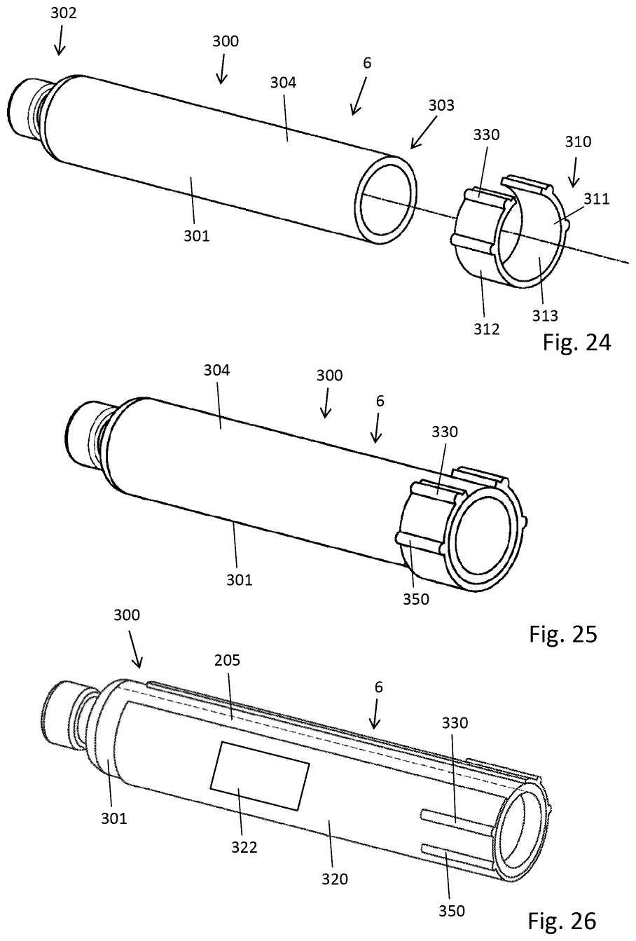

FIG. 24 is illustrative of a medicament container provided with a coding ring,

FIG. 25 shows the medicament container of FIG. 24 with the coding ring assembled to the medicament container, and

FIG. 26 shows another example of a medicament container provided with a label.

### DETAILED DESCRIPTION

In FIGS. 1 and 2 only one of numerous examples of a handheld injection device is illustrated, that is generally usable in combination with a wearable electronic device. The device as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 comprises a first housing component 100 and a second housing component 200. The second housing component may form a main housing part configured to accommodate a drive mechanism 8 and/or a dose setting mechanism 9 as shown in FIG. 2. The first housing component 100 is configured as a cartridge holder. It may be permanently or releasably connected to the second housing component 200.

The first housing component 100 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder and hence the first housing component 100 comprises a threaded socket 28 at its distal end to threadedly engage with a correspondingly threaded portion of the injection needle 15. By attaching the injection needle 15 to the distal end of the first housing component 100 the seal 26 of the cartridge 6 is penetrated thereby establishing a fluid transferring access to the interior of the cartridge 6.

When the injection device 1 is configured to administer e.g., human insulin, the dosage set by a dose dial 12 at a proximal end of the injection device 1 may be displayed in so-called international units (IU, wherein 1 IU is the biological equivalent of about 45.5 μg of pure crystalline insulin ( 1/22 mg). The dose dial 12 may comprise or may form a dose dial.

As shown further in FIGS. 1 and 2, the housing 10, e.g., the second housing component 200 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The click sound is typically generated by a click noise generator 45. Generally, a click noise generator 45 may be implemented in various different ways. The number sleeve 80 mechanically interacts with a piston in the insulin cartridge 6. When the needle 15 is stuck into a skin portion of a patient, and when the trigger 11 or injection button is pushed, the dose displayed in display window 13 will be ejected from injection device 1. When the needle 15 of the injection device 1 remains for a certain time in the skin portion after the trigger 11 is pushed, the dose is actually injected into the patient's body. Ejection of a dose of the liquid medicament may also cause a mechanical click sound, which is however different from the click sound produced when using the dose dial 12. For this, the injection device one may comprise a separate, hence a second click noise generator (not illustrated).

In this embodiment, during delivery of the insulin dose, the dose dial 12 is turned to its initial position in an axial movement, that is to say without rotation, while the number sleeve 80 is rotated to return to its initial position, e.g., to display a dose of zero units.

The injection device 1 may be used for several injection processes until either the cartridge 6 is empty or the expiration date of the medicament in the injection device 1 (e.g., 28 days after the first use) is reached.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10, e.g., to the second housing component 200. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement there between. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger sidewall or skirt extends from a periphery of the head and is further adapted to be seated in a proximally accessible annular recess of the dose dial 12.

To dial a dose a user rotates the dose dial 12. With the spring 40, also acting as a click noise generator 45, and the clutch 60 engaged, the drive sleeve 30, the spring 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g., in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may comprise at least one ratchet feature 91, such as a flexible arm on the sidewall of the tubular-shaped clutch 60. The at least one ratchet feature 91 may comprise a radially outwardly extending protrusion e.g., on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g., in WO2004/078239A1, WO 2004/078240A1 or WO 2004/078241A1 the entirety of which being incorporated herein by reference.

The housing 10 as illustrated in the numerous examples of FIGS. 3-26 comprises a first housing component 100 and a second housing component 200. The first housing component 100 is configured as a cartridge holder. It is sized and shaped to accommodate a medicament container 300 inside its hollow interior. The medicament container 300 may be implemented as a cartridge 6. The cartridge holder and hence the first housing component 100 comprises a first connecting end 101. The first connecting end 101 forms a proximal end of the first housing component 100. Correspondingly, the second housing component 200 comprises a second connecting end 201, typically at a distal end of the housing component 200.

The first connecting end 101 is mechanically connectable to the second connecting end 201. As illustrated, the first housing component 100 comprises an insert 110 forming the first connecting end 101. The second housing component 200 comprises a receptacle 210 shaped and sized to receive the insert 110. The receptacle 210 is radially confined by a sidewall 202 of the second housing component 200. The insert 110 is at least partially insertable into the receptacle 210 by a longitudinal sliding movement relative to the second housing component 200, in particular along the proximal direction 3.

The insert 110 forms a proximal end of the first housing component 100. The insert 110 comprises a proximal end face 112. Towards the distal direction 2 the insert 110 is confined by a flange section 115 protruding radially outwardly from the tubular shaped sidewall 102 of the first housing component 100 and hence also from a sidewall 102 of insert 110. The flange section 115 may be structurally stiffened by a circumferential protruding radially outwardly extending rim protruding from the outside surface 105 of the sidewall 102 of the first housing component 100.

Towards the proximal direction 3 the flange section 115 comprises an abutment or stop face 114 facing in proximal direction 3. The abutment or stop face 114 is configured to axially abut a distal end face 214 of a sidewall 202 of the second housing component 200. The sidewall 102 of the first housing component 100 may comprise a window 103, which can be implemented as a through recess intersecting the sidewall 102. The window 103 allows and supports visual inspection of the cartridge 6 and its content arranged inside the first housing component 100.

The receptacle 210 is open towards the distal direction 2. Insofar, the insert 110 of the first housing component 100 can be inserted into the receptacle 210 along the proximal direction 3 until a final assembly configuration has been reached. There is provided a fastening element 120 on the outside surface 105 of the insert 110 complementary shaped to a counter fastening element 220 supported on the sidewall 202 that confines the receptacle 210.

With the example of FIGS. 3-14 the fastening element 120 is provided on the insert 110. The fastening element 120 comprises a fastening projection 124. The fastening projection 124 may comprise a peg or pin-shaped radially outwardly extending projection protruding radially outwardly from an outside surface 105 of the first connecting end 101. The fastening projection 124 and hence the fastening element 120 may comprise a snap element 121. Complementary shaped on the inside surface of 203 of the receptacle 210 there is provided a counter fastening element 220. The counter fastening element 220 comprises a radial recess 222 in the inside surface 203 of the sidewall 202.

The counter fastening element 220 comprises a fastening groove 224. As particularly illustrated in FIG. 7 the fastening groove 224 comprises a first groove portion 225 and a second groove portion 226. The second groove portion 226 extends in circumferential direction (w). The first groove portion 225 extends in longitudinal direction (z). The second groove portion 226 merges into the first groove portion 225. The first groove portion 225 may extend perpendicular to the second groove portion 226. This way, a kind of a L-shaped fastening groove 224 may be provided that supports a kind of a bayonet connection for the fastening element 120 engaging the counter fastening element 220.

The counter fastening element 220 is provided with a counter snap element 221. The counter snap element 221 is complementary shaped to the snap element 121. As illustrated in FIG. 7 the counter snap element 221 comprises a radial protrusion and/or a radial recess at or near a dead end of the second groove portion 226 facing away the first groove portion 225. The shape and cross-section of the first groove portion 225 is complementary to the shape and cross-section of the radially outwardly protruding fastening projection 124.

During and for a mutual assembly of the first housing component 100 and the second housing component 200 the fastening projection 124 is aligned with the first groove portion 225. Then, and through a longitudinal displacement of the first housing component 100 in proximal direction 3 relative to the second housing component 200 the fastening projection 124 is guided into and through the first groove portion 225. Upon reaching a final insert position or insert configuration, e.g., when the stop face 114 engages and longitudinally abuts the distal end face 214 of the second housing component 200 the fastening projection 124 may be longitudinally or circumferentially aligned with the second groove portion 226. Then and through a twisting motion of the first housing component 100 relative to the second housing component 200 the fastening projection 124 slides along the second groove portion 226. The snap element 121 and hence the fastening projection 124 then mechanically engages the complementary shaped counter snap element 221 provided in the second groove portion 226.

As illustrated in FIGS. 7 and 14 there may be provided a twofold mutual engagement of a fastening element 120 with a respective counter fastening element 220. Hence, there may be provided a first fastening element 120 and a second fastening element 120 on diametrically opposite sides of the insert 110. Complementary, also the receptacle 210, at or near its insert opening 211 comprises a first and a second counter fastening element 220. Accordingly, respective first groove portions 225 of first and second counter fastening elements 220 adjoin the distal end face 214 of the sidewall 202 of the second housing component 200.

The shape of the fastening element 120 and counter fastening element 220 enables and supports a two-stage assembly process. During a first stage of assembly the first housing component 100 is exclusively subject to a longitudinal sliding movement relative to the second housing component 200. During the second stage of assembly and after reaching the final insert configuration the first housing component 100 is subject to a rotation relative to the second housing component.

The second stage of assembly is immediately apparent from a comparison of FIGS. 5 and 6. The first housing component 100 is provided with a visual indicator 108 on an outside surface 105. Complementary, the second housing component 200 is provided with a visual counter indicator 208 provided on the outside surface 205 of the sidewall 202. The indicator 108 is located distally offset from the flange section 115. The visual counter indicator 280 is provided at or near the second connecting end 201.

In FIG. 5, the first and second housing components 100, 200 are in the final insert configuration, in which the stop face 114 of the flange section 115 is in axial abutment with the distal end face 214 of the sidewall 202 of the second housing component 200. As indicated in FIG. 5, the indicator 108 is circumferentially offset from the counter indicator 208. Through a twisting motion of the first housing component 100 relative to the second housing component 200 the indicator 108 is brought in alignment with the complementary shaped counter indicator 208. In this way, a visual and/or palpable, hence haptically detectable feedback is provided to the user that the final assembly configuration has been reached.

The first housing component 100 comprises a tubular-shaped sidewall 102. The sidewall 102 radially narrows near a distal end and comprises a radially inwardly extending shoulder portion 106. Distally from the shoulder portion 106 may extend a further stepped socket portion, that may provide a detachable connection for a piercing assembly.

The interior of the first housing component 100 is sized and shaped to receive the medicament container 300. The medicament container 300 comprises a tubular-shaped barrel 301. The barrel 301 comprises a sidewall 304. The medicament container 300 is insertable into the first housing component 100 along the distal direction 2. The insert motion may be delimited by the radially narrowing shoulder 306 of the barrel 301 engaging with an inside of the shoulder portion 106 of the housing component. Additionally or alternatively, a distal facing end face 307, e.g., located at a distal end 302 of the medicament container 300 may get in longitudinal abutment with an inside of a radially inwardly extending end face 107 of the first housing component 100. In this way, an insert motion of the medicament container 300 into the first housing component 100 may be delimited.

As illustrated in greater detail in FIG. 9 the medicament container 300 is provided with an alignment element 330 and further with a mechanical coding 350. Here, the alignment element 330 comprises an alignment feature 331 protruding radially outwardly as an alignment projection 332 from the proximal end 303 of the barrel 301. In addition, the mechanical coding 350 comprises a mechanical coding feature 351. The mechanical coding feature 351 is implemented as a coding projection 352 protruding radially outwardly at the proximal end 303 of the barrel 301 of the medicament container 30.

Complementary to the mechanical coding 350 and the alignment element 330 the first housing component 100 comprises a counter alignment element 130. The counter alignment element 130 is complementary shaped to the alignment element 330. In the present example the counter alignment element 130 is located at a proximal end of the insert 110. It comprises a longitudinally extending radial recess on the inside surface of the insert 110. Alternatively, it may provide a through recess or slit in the proximal end face 112 of the sidewall 102 of the insert 110.

The counter alignment element 130 thus comprises a counter alignment feature 131 that matches in size and shape with the alignment feature 331. The counter alignment element 130 and hence the counter alignment feature 131 comprises an alignment recess 132. The longitudinal extent of the alignment recess 132 and the longitudinal position of the distal end of the alignment projection 332 may likewise delimit and define the distally directed insert motion of the medicament container 300 into the first housing component 100.

The first housing component 100 further comprises a through recess 152 that is larger than or matches in shape with the mechanical coding 350 as provided on the medicament container 300. The through recess 152 comprises a longitudinal slot featuring a certain circumferential width by way of which the coding projection 352 of the mechanical coding 350 may reach radially through the sidewall 102 of the first housing component 100. This enables a mechanical engagement of the mechanical coding 350 with a complementary shaped mechanical counter coding 250 as provided on the inside 203 of the receptacle 210.

An example of the mechanical counter coding 250 is shown in FIG. 8. The mechanical coding 250 may be circumferentially offset from the counter fastening element 220. The mechanical coding 250 comprises a mechanical coding feature 251. The mechanical coding 250 comprises a radial recess 252, hence a coding recess sized and shaped to engage the radial coding projection 352 of the medicament container 300. The counter coding 250, hence the radial recess 252 comprises a coding groove 254. The coding groove 254 comprises a first groove portion 255 and a second groove portion 256. The first groove portion 255 extends in longitudinal direction. The second groove portion 256 extends in circumferential direction. The second groove portion 256 merges into the first groove portion 255. The first groove portion 255 adjoins the distal end face 214 of the sidewall 202 of the receptacle 210.

When the medicament container 300 is correctly assembled inside the first housing component 100 the alignment feature 331 engages the counter alignment feature 131. Vice versa, inserting of the medicament container 300 into the first housing component 100 requires a correct alignment of the alignment feature 331 with the counter alignment feature 131.

The mutually corresponding pair of the alignment projection 332 with the alignment recess 130 serves to prevent a rotation of the medicament container 300 relative to the first housing component 100. In other words, and when reaching the preassembly configuration the medicament container 300 is rotationally locked to the first housing component 100. Accordingly and since the coding projections 352 of the mechanical coding 350 comprises a radial extent that is larger than the thickness of the sidewall 102 of the first housing component 100 the coding projection 352 extends radially outwardly through the sidewall 102 of the first housing component as illustrated in FIGS. 13 and 14.

Then and upon mutually assembling the first housing component 100 and the second housing component 300 with the medicament container 300 preassembled inside the first housing component 100 the coding projection 352 will be aligned with the first groove portion 255 of the counter coding groove 254. During the first stage of assembly and hence when the first housing component 100 is exclusively subject to a proximal sliding movement relative to the second housing component 200 the coding projection 352 slides along the first groove portion 255.

When reaching the final insert configuration, the coding projections 352 aligns with the complementary shaped second groove portion 256. Then, a mutual twisting of the first and second housing component 100, 200 becomes possible by way of which the fastening projection 125 slides along the second groove portion 226 and during which the coding projection 352 slides along the second groove portion 256.

In FIG. 11, the mechanical engagement of the fastening projection 124 and hence of the snap element 121 with the complementary-shaped counter snap element 221 is schematically illustrated. In FIG. 12, the location of the alignment element 330 and the mechanical coding 350 inside the second housing component 200 in the final assembly position is schematically illustrated without showing the first housing component 100.

In FIGS. 11 and 12 the second housing component 200 is represented by a housing insert 206. The housing insert 206 may be of tubular shape and may provide the receptacle 210 comprising the mechanical counter coding 250 and/or comprising the counter fastening element 220. The housing insert 206 may be rigidly fastened or integrally formed with a proximal housing part 207. Thus, when the second housing 200 comprises a proximal tubular shaped housing part 207, the housing insert 206 may be attached and fastened or fixed to a distal end of the proximal housing part. Otherwise, the second housing component 200 may be implemented as a single piece. Then, the geometric structure of the receptacle 210 is integrated into the elongated tubular shaped sidewall 202 of the second housing component 200.

The housing insert 206 may comprise a radially outwardly extending stepped portion at a distal end forming or constituting a rim 216. The rim 216 may enhance stability and rigidity of the receptacle 210 when attached to the tubular shaped proximal housing part 207. The housing insert 206 may be rigidly or permanently fastened to the proximal housing part 207. In this way, and for providing a set or a kit of different housing components configured for use with different medicament containers 300 only the housing insert 206 must be provided individually for a second housing component 200. For each housing 10 of a set or kit of differently encoded housings there may be used always the same proximal housing part 207 individually equipped with a suitable distinguishing housing insert 206.

Likewise, the first housing component 100 may not be subject to a mechanical encoding. Hence, for a kit or set of differently encoded housings or injection devices there may be always used one and the same first housing component 100, provided that the mechanical coding 350 as provided on the outside of the medicament container 300 is allowed to reach or extend through the first housing component 100. Here, the circumferential size or width of the through recess 152 may be increased to enable insertion of all available types of the mechanical coding 350 of differently configured medicament containers 300.

In this way, a set or kit of differently encoded injection devices can be provided on the basis of one and the same first housing component 100. Hence, one and the same first housing component 100 can be used for a set of differently encoded second housing components. With a kit of housings or injection devices it may be only and exclusively the medicament container 300 and the proximal, and hence the second housing components 200 that are subject to a particular mechanical encoding. The fastening element 120 as provided on the first housing component and the complementary-shaped counter fastening element 220 as provided on the second housing component 200 may be of equal shape for all available housings and injection devices of a kit or set of differently encoded housings or injection devices.

In FIG. 15, a first example of a mechanical coding 350 matching with a mechanical counter coding 250 is illustrated. Here, the first housing component 100 is provided with radially outwardly extending fastening element 120 matching in size and shape as well as with regard to the circumferential and radial position with a complementary-shaped counter fastening element 220 of the second housing components 200. The alignment element 330 and hence the alignment projection 332 of the medicament container 300 matches the complementary-shaped counter alignment element 130 and the respective alignment recess 132 as provided on the inside surface of the sidewall 102 of the first housing component 100.

The first housing component 100 further comprises the through recess 152 that matches in size and shape with the size and shape of the coding feature 351 and hence of the radially outwardly extending coding projection 351 as provided on the outside surface of the medicament container 300. Accordingly, the medicament container 300 can be longitudinally inserted into the first housing component 100, thereby aligning the alignment element 330 with the complementary-shaped counter alignment element 130. Correspondingly, the coding projection 152 may reach and extend radially through the through recess 152. Then and during the final assembly of the first and the second housing components 100, 200 the coding projection 352 aligns with the radial recess 252 of the complementary shaped counter coding feature 251 and the respective mechanical counter coding groove 254.

With the presently illustrated example the through recess 152 of the first housing component 100 may even provide a secondary mechanical counter coding 150. The secondary mechanical counter coding 150 comprises a counter coding feature 151 in form of the through recess 152. In this way insertion of a medicament container 300 into the first housing component 100 is only possible when the mechanical coding 350 matches the secondary mechanical counter coding 150. Otherwise, e.g., when the circumferential position of the mechanical coding 350 relative to the alignment element 330 does not match the circumferential position of the secondary mechanical counter coding 150 with regard to the counter alignment element 130, or alternatively, when the size and/or shape of the mechanical coding 350 does not match with the secondary mechanical counter coding 150 the medicament container 300 cannot be inserted into the first housing component 100. This provides additional safety for not assembling a medicament container 300 into a wrong first housing component 100.

In FIG. 16 another example of a mechanical coding 350′ is illustrated. Here, the mechanical coding 350′ varies from the mechanical coding 350 by the circumferential position of the mechanical coding feature 351′ and hence by the circumferential position of the coding projection 352′ with regard to the circumferential position of the alignment element 330.

Compared to the mechanical coding 350 of FIG. 15 the mechanical coding 350′ of FIG. 16 is rotated counterclockwise. Accordingly, also the secondary mechanical counter coding 150′ is shifted or rotated counterclockwise. There, the respective counter coding feature 151′ and hence the through recess 152′ allows a longitudinal insertion of the modified mechanical coding 350′.

Accordingly, also the mechanical counter coding 250′, the counter coding feature 251′ and hence the radial recess 252′ as provided on the second housing component 200 has changed accordingly. It is immediately apparent, that the medicament container 300 with the mechanical coding 350 of a first type and as illustrated in FIG. 15 cannot be inserted into the first housing component 100 provided with the secondary mechanical counter coding 151′ of the second type as illustrated in FIG. 16.

Moreover, the medicament container 300 with the mechanical coding 350′ of a second type correctly assembled inside the housing component 100 and provided with the secondary mechanical counter coding 150′ of the second type cannot be attached or connected to the second housing component 200 provided with the mechanical counter coding 250 of the first type as illustrated in FIG. 15. Here, the circumferential position of the coding feature 351′ does not match the circumferential position of the counter coding feature 251 with regards to the circumferential position of the counter fastening element 220.

With the further example of FIG. 17 the circumferential position of the alignment element 330′ of the medicament container 300 has been modified compared to the examples of FIGS. 15 and 16. Accordingly, the circumferential position of the counter alignment element 130′ relative to the counter fastening element 120 has been also varied compared to the position of the counter alignment element 130 relative to the counter fastening element 120 of the example of FIGS. 15 and 16. Moreover, the relative circumferential position of the mechanical coding 350″ and hence the circumferential position of the respective coding feature 351″ relative to the alignment element 330′ and the alignment projection 332′ has been amended compared to the relative circumferential position of the mechanical coding 350, 350′ relative to the alignment element 330 of the examples of FIG. 15 or 16.

As illustrated in FIGS. 15-17 the mechanical coding 350″ of a third type is exclusively insertable into the secondary mechanical counter coding 150″ as provided by the first housing component according to FIG. 17.

The preassembly of the medicament container 300 provided with the mechanical coding 350″ of the third type is exclusively connectable to the second housing component 200 that is provided with a complementary shape mechanical counter coding 250″ of the third type. The preassembly of the first housing component 100 and the medicament container 300 of FIG. 17 cannot engage or connect with any of the second housing components 200 provided with the mechanical counter coding 250 of the first type as illustrated in FIG. 15 or with the second housing component 200 provided with a mechanical counter coding 250′ of a second type as illustrated in FIG. 16.

In FIGS. 18 through 23 two further examples of a mechanical coding 350, 350′ and a complementary shaped mechanical counter coding 250, 250′ are illustrated. Here, and for illustration purpose the first housing component 100 is not particularly illustrated. The mechanical coding 350 of a first type is illustrated in FIGS. 18-20. The mechanical coding 350 comprises two coding features 351, each of which implemented as a radially outwardly extending coding projection 352 on the proximal end of the sidewall 304 of the medicament container 300. The proximal end of the coding projections 352 may adjoin or may flush with the proximal end face of the barrel 301. The coding projections 352 may comprise a longitudinally extending coding rib.

In FIG. 19, the complementary shaped counter coding 250 is illustrated. The mechanical counter coding 250 comprises a coding groove 254 with a first groove portion 255 and a second groove portion 256 as described above in greater detail. The receptacle 210 is delimited in proximal direction by a radially inwardly extending end face 112, e.g., formed by a flange portion of the housing insert 206. As illustrated, the second groove portion 256 longitudinally adjoins the end face 212. Upon reaching a final insert position the barrel 301 may longitudinally abut the end face 212 and the coding projection 352 may properly align the second groove portion 256.

With the further example of FIGS. 21-23 the mechanical coding 350′ comprises a coding feature 351 also in form of a coding projection 352′. Compared to the coding projection 352 of FIG. 18 the coding projection 352′ comprises a longitudinal extent smaller than the longitudinal extent of the coding projection 352. Moreover, the longitudinal position of the coding projections 352′ is slightly longitudinally offset from the free or proximal end of the sidewall 304 of the barrel 301. The coding projection 352′ is shifted in distal direction 2 compared to the coding projection 352.

Complementary and as illustrated in FIG. 22, the second groove portion 256 of the complementary shaped counter coding feature 251′ and hence of the counter coding recess 252 varies with regards to the position and longitudinal size of the second groove portion 256 of the counter coding feature 251 as illustrated in FIG. 19.

Again, and when the barrel 301 reaches its final insert configuration inside the receptacle 210 the coding feature 351′ aligns the counter coding feature 251′ and a twisting motion of the medicament container 300 relative to the second housing component 200 is enabled by a sliding motion of the coding projection 352′ in circumferential direction (w) along the second groove portion 256 of the coding recess 252′.

With a non-matching combination of a mechanical coding 350 of a first type as illustrated in FIG. 18 with the mechanical counter coding 250′ of the second type as illustrated in FIG. 22 a mutual engagement will not be possible. First of all, the longitudinal extent of the coding projection 352 of the mechanical coding 350 of the first type is larger than a respective elongation or width of the second groove portion 256 of the mechanical counter coding 250′ of the second type.

Here, it may be possible that the mechanical coding 350 is longitudinally inserted into and slides along the first groove portion 255 of the mechanical counter coding 250′ of the second type. But here, the coding projection 352 prevents reaching of a final insert position and further prevents a twisting motion of the medicament container 300 and hence of the first housing component 100 relative to the second housing component 200.

Vice versa, the mechanical coding 350′ of the second type is unable to pair or to mate with the mechanical counter coding 250 of the first type. Here, upon reaching a final insert position the mechanical coding 350′ of the second type does not and cannot properly align with the second groove portion 256 of the mechanical counter coding 250 of the first type. So when reaching the final insert configuration a twisting motion of the medicament container 300 relative to the second housing component 200 is blocked and impeded.

In the illustration of FIGS. 24-26 an example is shown on how to provide the medicament container 300 with an alignment element 130 and with a mechanical coding 350. Here, the alignment element 330 and the mechanical coding 350 are both implemented as radially outwardly protruding ribs on an outside surface 312 of a coding ring 310. The coding ring 310 comprises an annual body 311. The annular body 311 may comprise a closed annular structure. As illustrated in FIG. 24 the annular body 311 may also comprise a longitudinal slit, thereby increasing the elastic deformation capability of the coding ring 310. With its inside surface 313 the annular body 311 may get in form fitting engagement with the sidewall 304 of the medicament container 300. The coding ring 310 may be provided as a separate part attached to the barrel 301 of the medicament container 300 by use of an adhesive or by welding.

In the further example of FIG. 26, the mechanical coding 350 as well as the alignment element 330 are provided on a label 320. They may be integrated into the label 320. The label 320 may comprise an indicator 322. The indicator 322 may be provided with readable or visual information thus providing supplemental information about the use or constitution of the medicament located inside the barrel 301. Here, the alignment element 330 and the mechanical coding 350 are provided as radially outwardly protruding ribs on the label 320. The label 320 may be adhesively attached on the outside surface of the barrel 301.

In general, there may be numerous additional ways on how to provide a mechanical coding 350 and an alignment element 330 on the outside of a medicament container 300. With further examples more which are not illustrated here, the mechanical coding 350 and/or the alignment element 330 may be integrally formed with the sidewall 304 of the barrel 301. Here, the barrel 301 may be made of a thermoplastic material. It may be implemented as an injection molded component.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the projection and the groove or in connection with the fastening element and counter fastening element may be interchanged and may be thus provided and implemented in an inverted way compared to the presently shown examples.

### REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
20 piston rod
21 bearing
22 first thread
23 pressure foot
24 second thread
25 barrel
26 seal
28 threaded socket
30 drive sleeve
31 threaded section
32 flange
33 flange
35 last dose limiter
40 spring
60 clutch
62 insert piece
64 stem
80 number sleeve
81 groove
90 ratchet mechanism
91 ratchet feature
100 housing component
101 connecting end
102 sidewall
103 window
105 outside surface
106 shoulder portion
107 end face
108 indicator
110 insert
112 end face
114 stop face
115 flange section
120 fastening element
121 snap element
124 fastening projection
130 counter alignment element
131 counter alignment feature
132 alignment recess
150 mechanical counter coding
151 counter coding feature
152 through recess
200 housing component
201 connecting end
202 sidewall
203 inside surface
205 outside surface
207 housing part
208 indicator
210 receptacle
211 insert opening
212 end face
214 end face
216 rim
220 counter fastening element
221 counter snap element
222 radial recess
224 fastening groove
225 groove portion
226 groove portion
250 mechanical counter coding
251 counter coding feature
252 radial recess
254 coding groove
255 groove portion
256 groove portion
300 medicament container
301 barrel
302 distal end
303 proximal end
304 sidewall
306 shoulder portion
307 end face
310 coding ring
311 annular body
312 outside surface
313 inside surface
320 label
322 indicator
330 alignment element
331 alignment feature
332 alignment projection
350 mechanical coding
351 coding feature
352 coding projection

1.-15. (canceled)

16. A housing of a drug delivery device, the housing comprising:
a first housing component comprising a first connecting end provided with a fastening element, the first housing component being shaped and configured to accommodate a medicament container, the medicament container comprising a mechanical coding and an alignment element;
a second housing component configured to accommodate a drive mechanism of the drug delivery device and comprising a second connecting end provided with a counter fastening element complementary shaped to the fastening element, the second connecting end being connectable to the first connecting end;
a counter alignment element complementary shaped to the alignment element and provided in or on the first housing component; and
a mechanical counter coding provided in or on the second housing component;
wherein when the medicament container is arranged in the first housing component and when the mechanical coding does not match the mechanical counter coding, the mechanical coding and the mechanical counter coding are operable to prevent an engagement of the fastening element with the counter fastening element.

17. The housing according to claim 16, wherein the first connecting end comprises a first sidewall and wherein the second connecting end comprises a second sidewall, and wherein one of the first connecting end and the second connecting end comprises an insert, and wherein the other one of the first connecting end and the second connecting end comprises a receptacle complementary shaped to the insert, and wherein the insert is insertable into the receptacle along a longitudinal direction for mutually fastening the first housing component and the second housing component.

18. The housing according to claim 17, wherein the mechanical counter coding comprises at least one counter coding feature and wherein the mechanical counter coding is defined by at least one of:
a number of counter coding features on the second sidewall,
a circumferential position of the counter coding features relative to the counter fastening element,
a circumferential extent of the counter coding features,
a radial extent of the counter coding features on the second sidewall, and
a cross-sectional geometry or shape of the counter coding features in a plane transverse to a longitudinal direction.

19. The housing according to claim 17, wherein the insert comprises a through recess in one of the first sidewall or second sidewall, the through recess being sized and shaped to receive at least one of the mechanical coding or the mechanical counter coding radially therethrough.

20. The housing according to claim 19, wherein the through recess provides or forms a secondary mechanical counter coding complementary shaped to the mechanical coding of the medicament container or complementary shaped to the mechanical counter coding of the second housing component.

21. The housing according to claim 20, wherein when the secondary mechanical counter coding does not match the mechanical coding or mechanical counter coding, the mechanical coding and the secondary mechanical counter coding are operable to prevent at least one of a complete insertion of the medicament container into the first housing component or a complete insertion of the insert into the receptacle.

22. The housing according to claim 16, wherein one of the fastening element and the counter fastening element comprises a fastening projection, and wherein the other one of the fastening element and the counter fastening element comprises a fastening recess.

23. The housing according to claim 22, wherein the fastening projection comprises a snap element, and wherein the fastening recess comprises a counter snap element complementary shaped to the snap element.

24. The housing according to claim 22, wherein the fastening recess comprises a fastening groove comprising a first groove portion and a second groove portion, wherein the first groove portion extends along a longitudinal direction and wherein the second groove portion extends along a circumferential direction and merges into the first groove portion.

25. The housing according to claim 16, wherein one of the mechanical coding and the mechanical counter coding comprises a radial coding projection, and wherein the other one of the mechanical coding and the mechanical counter coding comprises a coding recess complementary shaped to the radial coding projection.

26. The housing according to claim 25, wherein the coding recess comprises a coding groove comprising a first groove portion and a second groove portion, wherein the first groove portion extends along a longitudinal direction and wherein the second groove portion extends along a circumferential direction and merges into the first groove portion.

27. The housing according to claim 16, wherein one of the alignment element and the counter alignment element comprises an alignment projection, and wherein the other one of the alignment element and the counter alignment element comprises an alignment recess complementary shaped to the alignment projection.

28. A medicament container comprising:
a barrel sized and configured for insertion into a first housing component of a housing of a drug delivery device, wherein the housing comprises:
the first housing component comprising a first connecting end provided with a fastening element, the first housing component being shaped and configured to accommodate the medicament container, the medicament container comprising a mechanical coding and an alignment element,
a second housing component configured to accommodate a drive mechanism of the drug delivery device and comprising a second connecting end provided with a counter fastening element complementary shaped to the fastening element, the second connecting end being connectable to the first connecting end,
a counter alignment element complementary shaped to the alignment element and provided in or on the first housing component, and
a mechanical counter coding provided in or on the second housing component, wherein when the medicament container is arranged in the first housing component and when the mechanical coding does not match the mechanical counter coding, the mechanical coding and the mechanical counter coding are operable to prevent an engagement of the fastening element with the counter fastening element;
the alignment element on the barrel sized and shaped to engage with the counter alignment element of the first housing component when the barrel is inserted in a longitudinal direction into the first housing component; and
the mechanical coding on the barrel sized and shaped to engage with the mechanical counter coding of the second housing component when the first housing component with the barrel arranged inside is connected to the second housing component.

29. The medicament container according to claim 28, wherein the mechanical coding comprises at least one coding feature and wherein the mechanical coding is defined by:
a number of the at least one coding feature on the barrel,
a circumferential position of the at least one coding feature relative to the alignment element on the barrel,
a circumferential extent of the at least one coding feature,
a radial extent of the at least one coding feature on the barrel, and
a cross-sectional geometry or shape of the at least one coding feature in a plane transverse to the longitudinal direction.

30. An injection device comprising:
a housing of a drug delivery device, the housing comprising:
a first housing component comprising a first connecting end provided with a fastening element, the first housing component being shaped and configured to accommodate a medicament container, the medicament container comprising a mechanical coding and an alignment element,
a second housing component configured to accommodate a drive mechanism of the drug delivery device and comprising a second connecting end provided with a counter fastening element complementary shaped to the fastening element, the second connecting end being connectable to the first connecting end,
a counter alignment element complementary shaped to the alignment element and provided in or on the first housing component, and
a mechanical counter coding provided in or on the second housing component, wherein when the medicament container is arranged in the first housing component and when the mechanical coding does not match the mechanical counter coding, the mechanical coding and the mechanical counter coding are operable to prevent an engagement of the fastening element with the counter fastening element;
the medicament container comprising:
a barrel sized and configured for insertion into the first housing component of the housing,
the alignment element on the barrel sized and shaped to engage with the counter alignment element of the first housing component when the barrel is inserted in a longitudinal direction into the first housing component, and
the mechanical coding on the barrel sized and shaped to engage with the mechanical counter coding of the second housing component when the first housing component with the barrel arranged inside is connected to the second housing component, wherein the medicament container is arranged inside the first housing component; and
the drive mechanism arranged inside the second housing component.

31. The injection device according to claim 30, wherein the mechanical coding comprises at least one coding feature and wherein the mechanical coding is defined by:
a number of the at least one coding feature on the barrel,
a circumferential position of the at least one coding feature relative to the alignment element on the barrel,
a circumferential extent of the at least one coding feature,
a radial extent of the at least one coding feature on the barrel, and
a cross-sectional geometry or shape of the at least one coding feature in a plane transverse to the longitudinal direction.

32. A kit of injection devices, the kit comprising a first injection device and a second injection device,
wherein the first injection device comprises:
a housing of a drug delivery device, the housing comprising:
a first housing component comprising a first connecting end provided with a fastening element, the first housing component being shaped and configured to accommodate a medicament container, the medicament container comprising a mechanical coding and an alignment element,
a second housing component configured to accommodate a drive mechanism of the drug delivery device and comprising a second connecting end provided with a counter fastening element complementary shaped to the fastening element, the second connecting end being connectable to the first connecting end,
a counter alignment element complementary shaped to the alignment element and provided in or on the first housing component, and
a mechanical counter coding provided in or on the second housing component, wherein when the medicament container is arranged in the first housing component and when the mechanical coding does not match the mechanical counter coding, the mechanical coding and the mechanical counter coding are operable to prevent an engagement of the fastening element with the counter fastening element;
the medicament container comprising:
a barrel sized and configured for insertion into the first housing component of the housing,
the alignment element on the barrel sized and shaped to engage with the counter alignment element of the first housing component when the barrel is inserted in a longitudinal direction into the first housing component, and
the mechanical coding on the barrel sized and shaped to engage with the mechanical counter coding of the second housing component when the first housing component with the barrel arranged inside is connected to the second housing component, wherein the medicament container is arranged inside the first housing component; and
the drive mechanism arranged inside the second housing component;
wherein the second injection device comprises:
a further housing of a further drug delivery device, the further housing comprising:
a further first housing component comprising a further first connecting end provided with a further fastening element, the further first housing component being shaped and configured to accommodate a further medicament container, the further medicament container comprising a further mechanical coding and a further alignment element,
a further second housing component configured to accommodate a further drive mechanism of the further drug delivery device and comprising a further second connecting end provided with a further counter fastening element complementary shaped to the further fastening element, the further second connecting end being connectable to the further first connecting end,
a further counter alignment element complementary shaped to the further alignment element and provided in or on the further first housing component, and
a further mechanical counter coding provided in or on the further second housing component, wherein when the further medicament container is arranged in the further first housing component and when the further mechanical coding does not match the further mechanical counter coding, the further mechanical coding and the further mechanical counter coding are operable to prevent an engagement of the further fastening element with the further counter fastening element;
the further medicament container comprising:
a further barrel sized and configured for insertion into the further first housing component of the further housing,
the further alignment element on the further barrel sized and shaped to engage with the further counter alignment element of the further first housing component when the further barrel is inserted in a further longitudinal direction into the further first housing component, and
the further mechanical coding on the further barrel sized and shaped to engage with the further mechanical counter coding of the further second housing component when the further first housing component with the further barrel arranged inside is connected to the further second housing component, wherein the further medicament container is arranged inside the further first housing component; and
the further drive mechanism arranged inside the further second housing component;
wherein one of the mechanical coding and the mechanical counter coding of the first injection device distinguishes from the further mechanical coding or the further mechanical counter coding of the second injection device with regard to at least one of:
a number of coding features or counter coding features,
a circumferential position of the coding features relative to the alignment element on the barrel,
a circumferential position of the counter coding features relative to the counter fastening element,
a circumferential extent of the coding features or the counter coding features,
a radial extent of the coding features or the counter coding features, and
a cross-sectional geometry or shape of the coding features or the counter coding features in a plane transverse to the longitudinal direction.
